(19) United States
(12) Patent Application Publication
(10) Pub. No.: US 2017/0115247 A1
(43) Pub. Date: Apr. 27, 2017

(54) APPARATUS, METHOD AND SYSTEM FOR MEASURING ELECTRICAL PROPERTIES OF BIOLOGICAL SAMPLE AND METHOD FOR ASSESSING DEGREE OF AGING OF BIOLOGICAL SAMPLE

(71) Applicant: GWANGJU INSTITUTE OF SCIENCE AND TECHNOLOGY, Gwangju (KR)

(72) Inventors: Jong-Hyun LEE, Gwangju (KR); Jung-Joon CHA, Gwangju (KR); Yang Kyu PARK, Gwangju (KR)

(21) Appl. No.: 15/332,535

(22) Filed: Oct. 24, 2016

(30) Foreign Application Priority Data
Oct. 23, 2015 (KR) 10-2015-0148327

(51) Int. Cl. G01N 27/04; G01N 33/483

(52) U.S. Cl. CPC G01N 27/04; G01N 33/4833

(57) ABSTRACT

An apparatus for measuring electrical properties of a biological sample. The apparatus includes: a first channel through which a biological sample flows; a second channel formed above or below the first channel; and a plurality of electrodes adjoining the first channel and measuring electrical properties of the biological sample.

This application claims the benefit of Korean Patent Application No. 10-2015-0148327, filed on Oct. 23, 2015, entitled “Apparatus, method and system for measuring electronic characteristic of biomaterial and method for evaluating aging of biomaterial”, which is hereby incorporated by reference in its entirety into this application.

### BACKGROUND

1. Technical Field

The present invention relates to an apparatus, method, and system for measuring electrical properties of a biological sample which can measure a difference in impedance caused by age-related changes in components of a cell or tissue, thereby assessing a degree of aging of the cell or tissue, and a method for assessing a degree of aging of a biological sample.

2. Description of the Related Art

All living organisms undergo weakening of the immune system and are more likely to catch a disease with age. If the difference in mechanism between aging and disease can be clearly elucidated, such elucidation will provide a potential effect on diagnosis of disease and proper therapeutic planning.

As indices of aging, functional changes such as kidney/liver/lung functionality, metabolism/immunity, blood cholesterol, blood pressure, tooth/eye/heart health, or physical/mental functionality; or biomarkers observable in a living body, such as a degree of intracellular activation of enzyme, relation between aging and accumulation of reactive oxygen species (ROS), changes in length of telomeres of chromosomes with age, and polyunsaturated fatty acid (PUFA) are mainly utilized.

However, such functional changes are an indirect and relative index of aging, and biomarkers have a problem in that detection thereof is very complex and difficult to conduct, despite being a direct index of aging.

In “Quantification of biological aging in young adults” (Daniel W. Belsky et al, Proceedings of the National Academy of Sciences of the United States of America, June 2015), aspects of change in various biomarkers and functionality with age are utilized to elucidate aging mechanisms.

### BRIEF SUMMARY

It is an aspect of the present invention to provide an apparatus, method, and system for measuring electrical properties of a biological sample which can measure a difference in impedance caused by age-related changes in components of a cell or tissue, thereby assessing a degree of aging of the cell or tissue, and a method for assessing a degree of aging of a biological sample.

In accordance with one aspect of the present invention, an apparatus for measuring electrical properties of a biological sample may include: a first channel through which a biological sample flows; a second channel formed above or below the first channel; and a plurality of electrodes adjoining the first channel and measuring electrical properties of the biological sample.

A portion of a surface of the first channel between the first channel and the second channel may be formed of a flexible film.

The flexible film may be deformed by a pressure of the second channel.

The flexible film may be bent inwardly of the first channel with increasing pressure of the second channel.

As the flexible film is bent inwardly of the first channel, the electrodes may be brought into contact with the biological sample in the first channel.

The first channel may be provided therein with a biological sample trap.

The biological sample trap may include a plurality of barriers, and the barriers may be separated from one another by a distance corresponding to the size of a single biological sample.

The electrodes may measure electrical properties of the biological sample, and the electrical properties may include at least one of amplitude, phase, reactance, resistance, conductance, and capacitance.

The biological sample may include at least one of a cell, a tissue, and an organ of a living organism.

The first channel, the second channel, or the biological sample trap may be formed by lithography and photoresist processes.

The apparatus may further include: a controller which regulates an pneumatic pressure of the second channel when a single biological sample is situated in the biological sample trap.

The first channel may be orthogonal to the second channel.

The second channel may be a negative pressure channel and the first channel may communicate with the second channel at a position at which the biological sample trap is provided.

The biological sample trap may include a plurality of barriers inclined in a direction in which the first channel communicates with the second channel.

The electrodes may be formed on the barriers.

The barriers may have an inclined structure.

In accordance with another aspect of the present invention, a system for measuring electrical properties of a biological sample may include: the apparatus for measuring electrical properties of a biological sample as set forth above; an electrical property analyzer analyzing electrical properties measured by the apparatus; and a pneumatic pump infusing air into the second channel.

The system may further include: a display displaying results of measuring and analyzing the electrical properties.

The system may further include: a computing device storing output values of the electrical property analyzer and assessing a degree of aging of the biological sample based on the stored output values.

In accordance with a further aspect of the present invention, a method for measuring electrical properties of a biological sample using the apparatus as set forth above may include: determining whether a single biological sample is situated in the biological sample trap; and increasing a pressure of the second channel upon determining that the single biological sample is situated in the biological sample trap.

In accordance with yet another aspect of the present invention, a method for assessing a degree of aging of a biological sample includes: measuring electrical properties of a plurality of biological samples using the apparatus as set forth above; calculating a difference between the measured electrical properties of the biological samples; and assessing a degree of aging of the biological samples based on the measured electrical properties and the calculated difference.

According to one embodiment of the present invention, changes in impedance of a cell with age can be measured, whereby a degree of aging can be digitized and quantified without using other biomarkers.

Thus, it is possible to overcome limitations in elucidation of aging mechanisms of typical assessment methods which utilize functional assessment and biomarkers, thereby presenting a new criterion of aging.

In addition, it is possible to provide a direct measurement method which can exhibit a relatively high correlation with aging mechanisms, as compared with a typical indirect measurement method such as functional assessment.

Further, it is possible to assess a degree of aging in a faster and simpler manner than a chemical assessment method using biomarkers while minimizing the risk of errors using digitized and quantified results obtained by instrumental measurement.

### BRIEF DESCRIPTION OF THE DRAWINGS

The above and other aspects, features, and advantages of the present invention will become apparent from the detailed description of the following embodiments in conjunction with the accompanying drawings, in which;

FIGS. 1a and 1b are schematic views of an apparatus for measuring electrical properties of a biological sample according to one embodiment of the present invention;

FIGS. 2a, 2b and 3 are views illustrating a mechanism of the apparatus for measuring electrical properties of a biological sample according to one embodiment of the present invention;

FIG. 4 is a schematic diagram of a system for measuring electrical properties of a biological sample according to one embodiment of the present invention;

FIGS. 5a to 10c are graphs output from the system for measuring electrical properties of a biological sample according to one embodiment of the present invention; and

FIGS. 11a to 11c are views of an apparatus for measuring electrical properties of a biological sample according to another embodiment of the present invention.

### DETAILED DESCRIPTION

The particular structural or functional descriptions of embodiments according to the concepts of the present invention disclosed in the specification or the application are only intended for the purpose of describing embodiments according to the concepts of the present invention and the embodiments according to the concepts of the present invention may be practiced in various forms and should not be construed as being limited to those described in the specification or the application.

The present invention may be realized by various embodiments, and some exemplary embodiments of the present invention will be described in detail with reference to the accompanying drawings. However, it should be understood that the present invention is not limited to the following embodiments, and that various modifications, substitutions, and equivalent embodiments can be made by those skilled in the art without departing from the spirit and scope of the present invention.

Although the terms first, second, etc. may be used herein to describe various elements, components, regions, layers, and/or sections, these elements, components, regions, layers, and/or sections should not be limited by these terms. These terms are used to distinguish one element, component, region, layer, and/or section from another element, component, region, layer, and/or section. Thus, a first element, component, region, layer, and/or section discussed below could be termed a second element, component, region, layer, and/or section without departing from the teachings of the present disclosure.

When an element or layer is referred to as being “on,” “connected to,” or “coupled to” another element or layer, it may be directly on, connected to, or coupled to the other element or layer or intervening elements or layers may be present. When, however, an element or layer is referred to as being “directly on,” “directly connected to,” or “directly coupled to” another element or layer, there are no intervening elements or layers present. Other expressions describing the relationship between components, such as “between” and “directly between” or “adjacent to” and “adjacent directly to” should also be construed in the same way.

The terminology used herein is for the purpose of describing particular embodiments and is not intended to be limitative. As used herein, the singular forms, “a,” “an,” and “the” are intended to include the plural forms as well, unless the context clearly indicates otherwise. Moreover, the terms “comprises,” “comprising,” “includes,” and/or “including,” when used in this specification, specify the presence of stated features, integers, steps, operations, elements, components, and/or groups thereof, but do not preclude the presence or addition of one or more other features, integers, steps, operations, elements, components, and/or groups thereof.

Unless otherwise defined herein, all terms including technical or scientific terms used herein have the same meanings as commonly understood by those skilled in the art to which the present invention pertains. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein.

Hereinafter, embodiments of the present invention will be described with reference to the accompanying drawings. It should be noted that like components will be denoted by like reference numerals throughout the specification and the accompanying drawings.

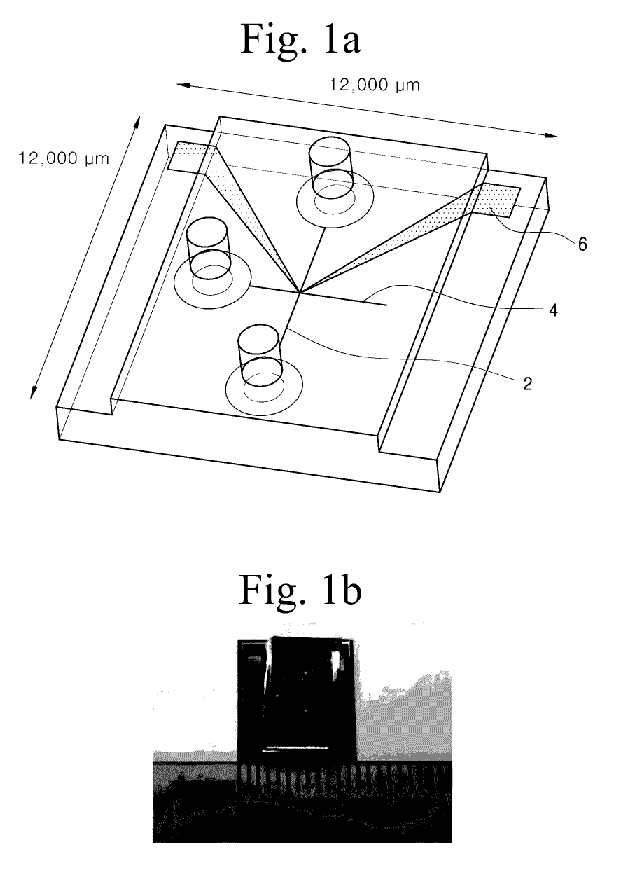

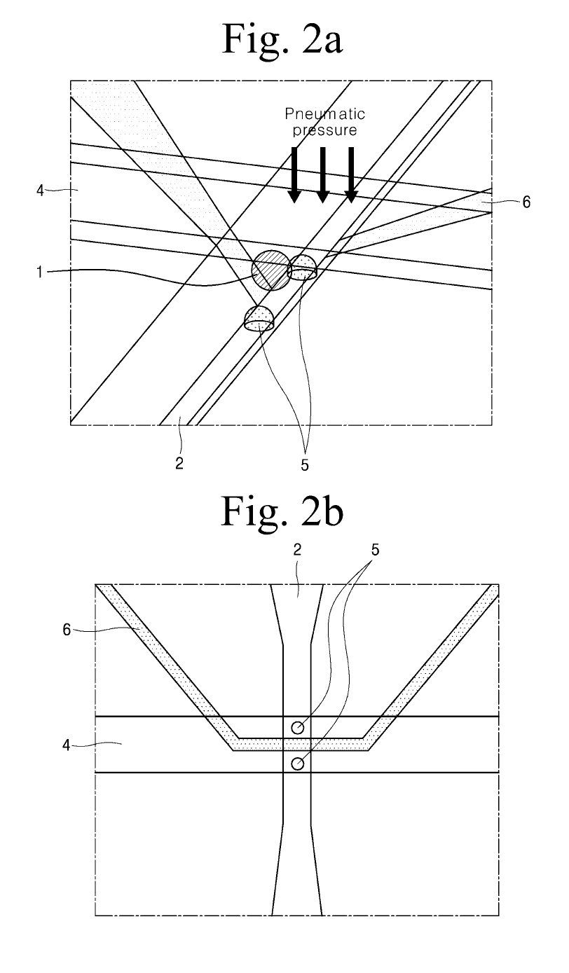

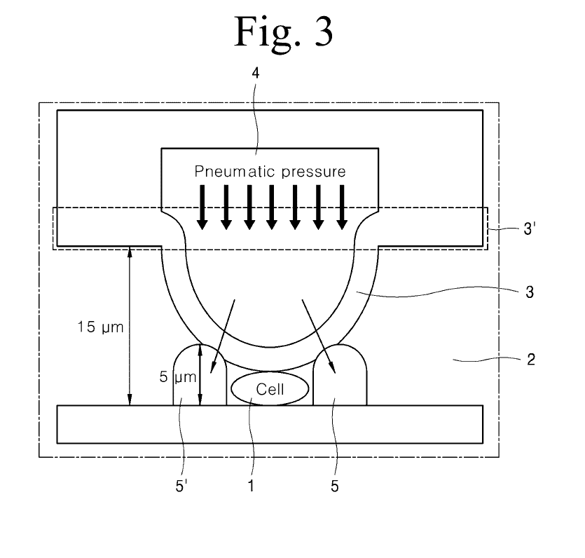

FIGS. 1a and 1b are schematic views of an apparatus for measuring electrical properties of a biological sample according to one embodiment of the present invention, and FIGS. 2a, 2b and 3 are views illustrating a mechanism of the apparatus for measuring electrical properties of a biological sample according to one embodiment of the present invention.

Referring to FIGS. 1a, 1b, 2a, 2b and 3, the apparatus for measuring electrical properties of a biological sample according to this embodiment may include: a first channel 2 which is a fluidic channel through which a biological sample flows; a second channel 4 which is a pneumatic channel having an air-pressure adjustable function and is formed above or below the first channel 2; and a plurality of electrodes 6 adjoining the first channel 2 and sensing electrical properties of the biological sample.

The biological sample is introduced into the first channel 2.

Electrical properties of the biological sample may include at least one of amplitude, phase, reactance, resistance, conductance, and capacitance. In addition, the biological sample may include at least one of a cell, a tissue, and an organ of a living organism.

The first channel 2 and the second channel 4 may be formed by lithography and photoresist processes. The pressure of the second channel 4, which is a pneumatic channel, may be increased and decreased by a separate controller (not shown). The controller may be a CPU, a microprocessor, or a microcontroller, and may be realized as software or firmware. The controller may be composed of a plurality of sub-controllers, as needed.

A portion of a surface of the first channel 2 between the first channel 2 and the second channel 4 may be formed of flexible films 3, 3′. The flexible films 3, 3′ may be deformed by change in pressure of the second channel 4. That is, when pneumatic pressure is applied to the second channel 4, which is a pneumatic channel, the flexible films 3, 3′ are deformed to reduce the height of the fluidic channel through which the biological sample flows, such that the biological sample can be brought into contact with the electrodes 6.

In other words, the flexible film 3 connected to the second channel 4 is bent inwardly of the first channel 2 with increasing pressure of the second channel 4, and the flexible film 3′ not connected to the second channel 4 is not deformed regardless of increase in pressure of the second channel 4. As the flexible film 3 is bent inwardly of the first channel 2, the electrodes 6 capture a single biological sample from biological samples flowing through the first channel 2. To this end, a biological sample trap is formed inside the first channel 2.

That is, the first channel 2 may be provided therein with the biological sample trap. The biological sample trap may include a plurality of barriers 5, 5′. The barriers 5, 5′ are separated from one another by a distance corresponding to the size (for example, width or height) of a single biological sample.

Referring to FIG. 3, the first channel 2 may have an inner height of about 15 μm and the barriers 5, 5′ may have a height of 5 μm, without being limited thereto. The barriers 5, 5′ between the electrodes 6 are configured to trap a single biological sample without interference by other biological samples, for example, a next cell.

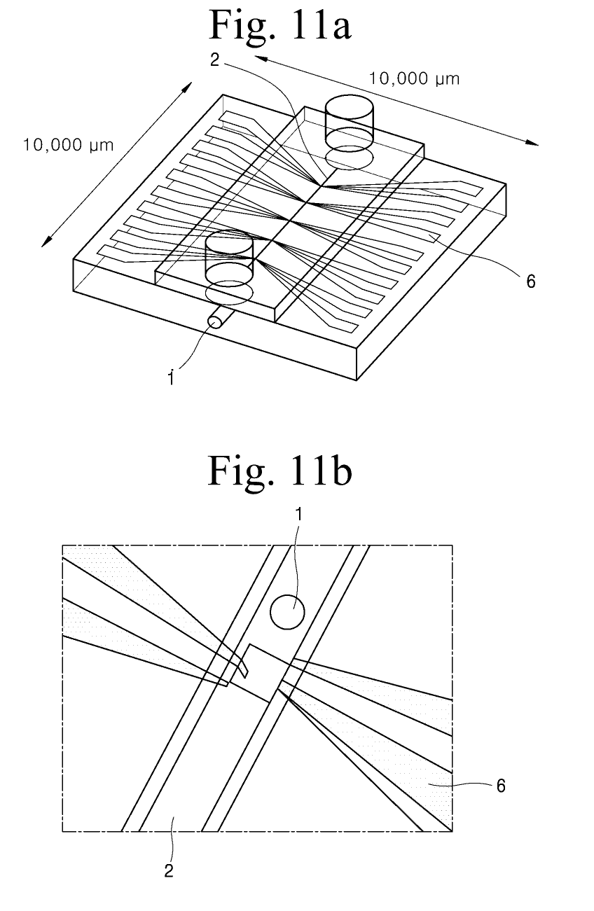

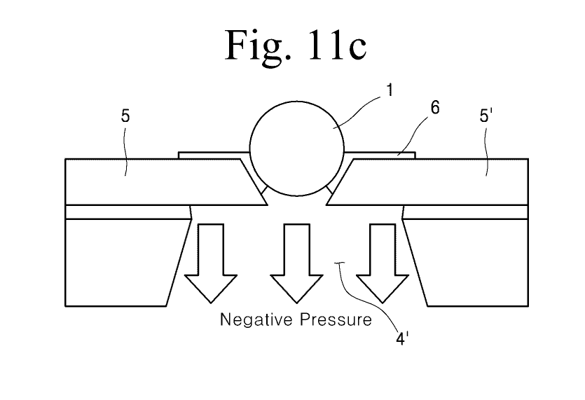

The first channel 2 may be orthogonal to the second channel 4 or may be parallel to the second channel 4. In addition, a separate third channel (not shown) may be further provided to be orthogonal or parallel to the first channel 2. The third channel may be a negative pressure channel described below with reference to FIGS. 11a to 11c.

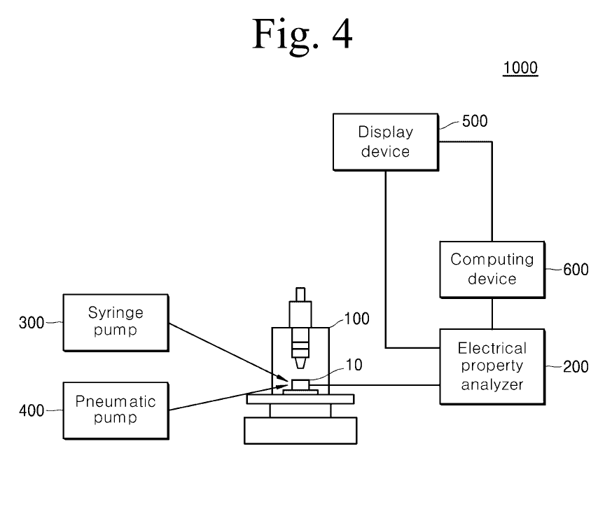

FIG. 4 is a schematic diagram of a system for measuring electrical properties of a biological sample according to one embodiment of the present invention. Referring to FIG. 4, the system 1000 for measuring electrical properties of a biological sample according to this embodiment of the present invention may include: a microscope 100 including the apparatus 10 for measuring electrical properties of a biological sample; an electrical property analyzer 200; a syringe pump 300, a pneumatic pump 400, a display 500, and a computing device 600.

The system 1000 may be realized by connecting the apparatus 10 for measuring electrical properties of a biological sample as set forth above to other auxiliary devices capable of visualizing, analyzing, statistically interpreting, and assessing the measurement results in a wired or wireless manner.

Specifically, the system 1000 may include: an electrical property analyzer 200 analyzing electrical properties of a biological sample measured by the apparatus 100 for measuring electrical properties of a biological sample; a pneumatic pump 400 infusing air into the second channel 4; a computing device 600 storing a program for executing calculation, statistical interpretation, and assessment with respect to results of analysis by the electrical property analyzer 200; and a display 500 displaying results of analysis by the electrical property analyzer 200 and the computing device 600.

The computing device 600 may include a storage unit storing output values of the electrical property analyzer 200 and may assess a degree of aging of each biological sample based on the stored output values.

Here, the process of measurement and assessment may be performed by the following operations.

First, whether a single biological sample is situated in the biological sample trap is determined, and, upon determining that the single biological sample is situated in the biological sample trap, the pressure of the second channel 4 is increased such that the electrodes can be brought into contact with the biological samples to measure electrical properties of a plurality of biological samples. Alternatively, as soon as a single biological sample is situated in the biological sample trap, the electrodes formed on the barriers may be brought into contact with the biological sample to measure electrical properties.

Then, a difference between the measured electrical properties of the biological samples is calculated, and a degree of aging of each of the biological samples may be assessed based on the measured electrical properties and the calculated difference. Here, the degree of aging may be assessed using a database created based on experimental results as described below.

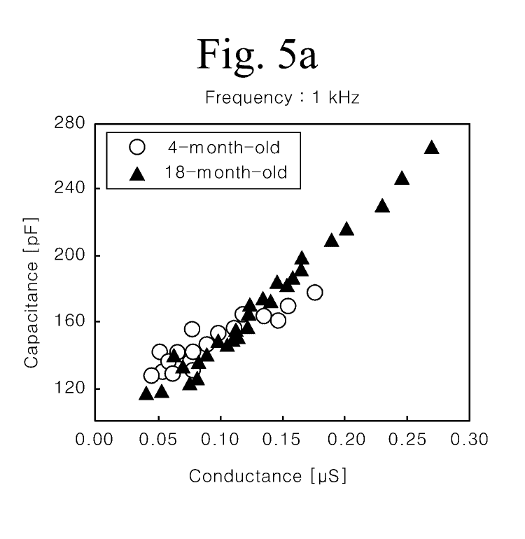

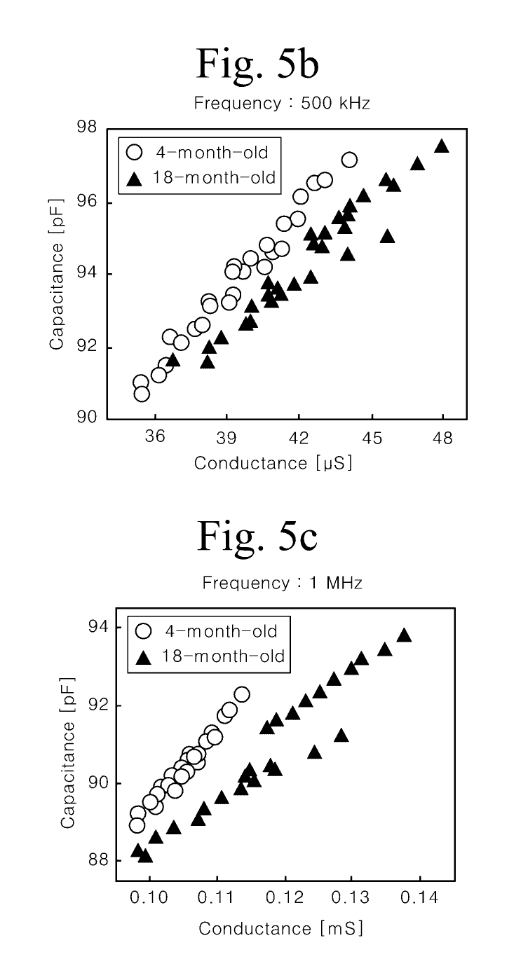

FIGS. 5a to 10c are graphs output from the system for measuring electrical properties of a biological sample according to one embodiment of the present invention.

FIGS. 5a to 5c are graphs showing changes in capacitance vs. conductance of bio-signals with varying frequency. In this experiment, cells of 3 months old, 4 months old, and 18 months old zebrafish (hereinafter, referred to as a first cell, a second cell, and a third cell, respectively) were used, and electrical properties of the cells were measured at a frequency of 10 kHz, 100 kHz, and 1 MHz.

Specifically, in this experiment, vascular endothelial cells extracted from dissected hearts of transgenic zebrafish were isolated by enzyme-cell separation and fluorescence-activated cell sorting. Then, electrical properties of the vascular endothelial cells were measured using the system for measuring electrical properties of a biological sample according to one embodiment of the invention, thereby obtaining the above graphs.

Generally, the lifespan of zebrafish is 2 years and zebrafish are mature when they are 3 months old. It is thought that the results of this experiment are due to changes in fluidity of cells resulting from reduction in homeostasis for maintenance of balance between damage and recovery of the cells.

It was confirmed that the first cell, the second cell, and the third cell were considerably different from one another in terms of magnitude and phase of impedance. Specifically, comparing the first cell with the second cell, the second cell had a higher impedance than the first cell, and, comparing the second cell with the third cell, the third cell had a lower impedance than the second cell. In this experiment, the apparatus for measuring electrical properties of a biological sample according to the present invention succeeded in trapping a single cell with a success rate of 90%.

Referring to FIGS. 5a to 5c, it can be seen that a difference in electrical properties between the first to third cells becomes more evident with increasing frequency.

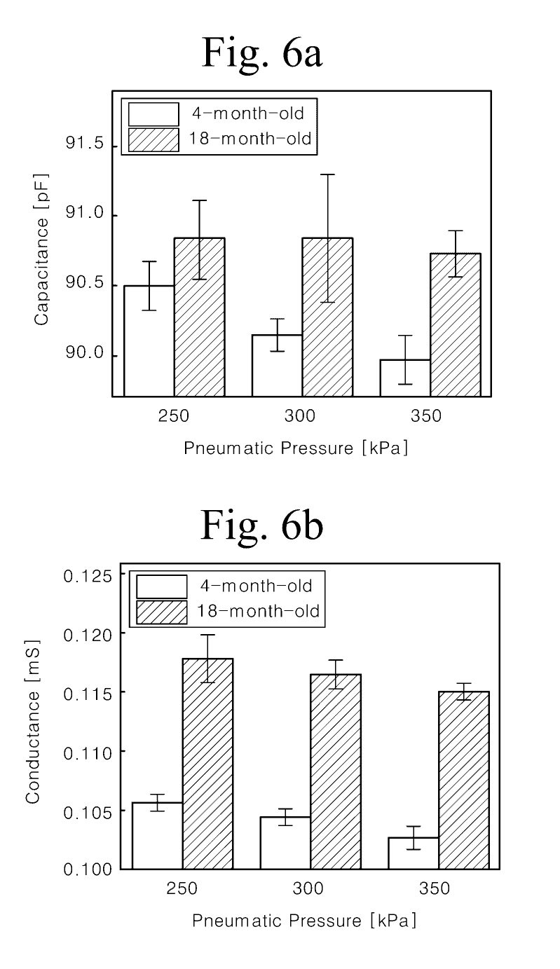

FIGS. 6a to 6b are graphs showing capacitance and conductance values, as measured at different pneumatic pressures for each cell. Specifically, FIGS. 6a to 6b show capacitance and conductance values, as measured at a pneumatic pressure of 250 kPa, 300 kPa, and 350 kPa and at a frequency of 1 MHz for each cell. In the graphs, each of the I-shaped graphs shows the standard error of the mean. Referring to FIGS. 6a to 6b, it can be seen that the capacitance and conductance values become larger with increasing age of the cells.

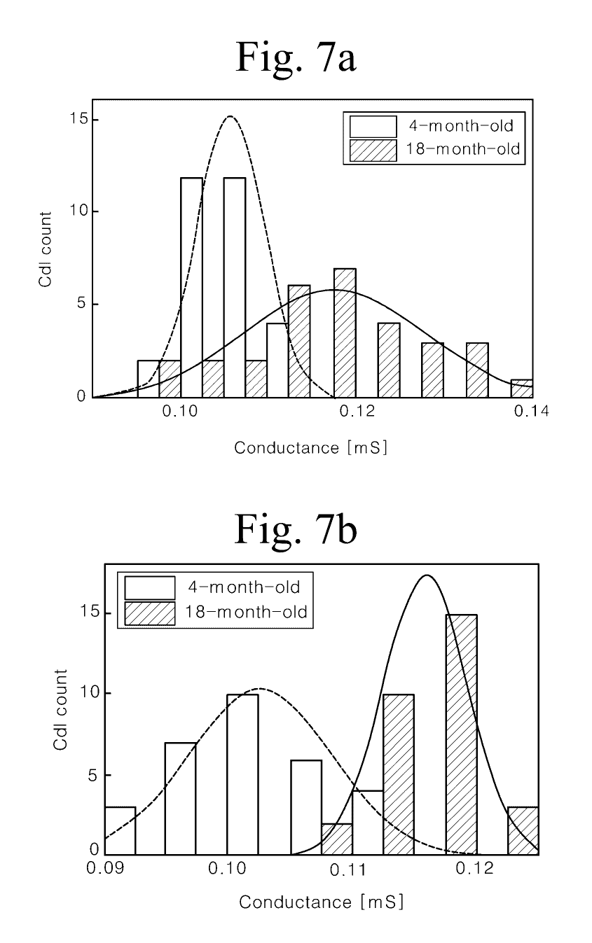

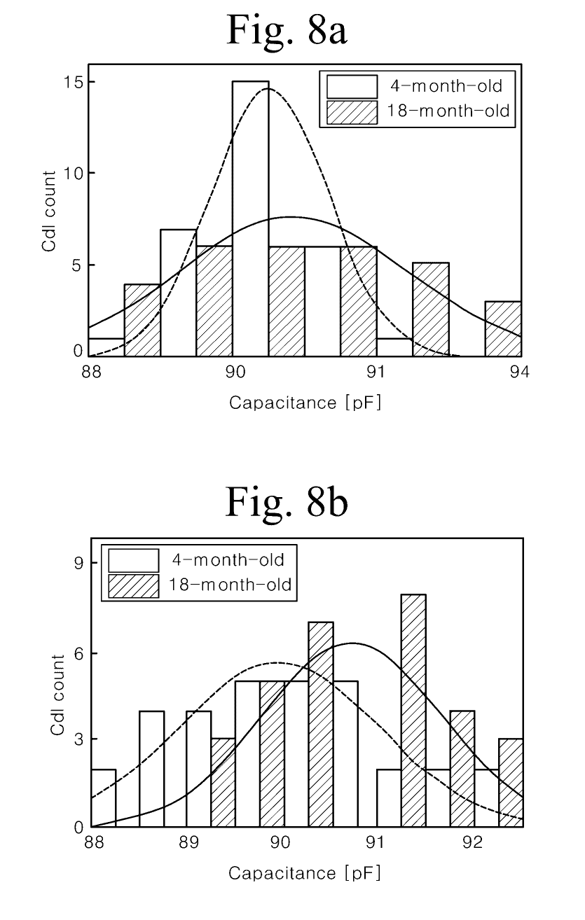

FIGS. 7a, 7b, 8a and 8b are histograms showing changes in impedance with varying magnitude of pneumatic pressure. FIG. 7a shows conductance values measured at a pneumatic pressure of 250 kPa, FIG. 7b shows conductance values measured at a pneumatic pressure of 350 kPa, FIG. 8a shows capacitance values measured at a pneumatic pressure of 250 kPa, and FIG. 8b shows capacitance values measured at a pneumatic pressure of 350 kPa. It can be seen that a difference in the values between the cells becomes more evident with increasing pneumatic pressure.

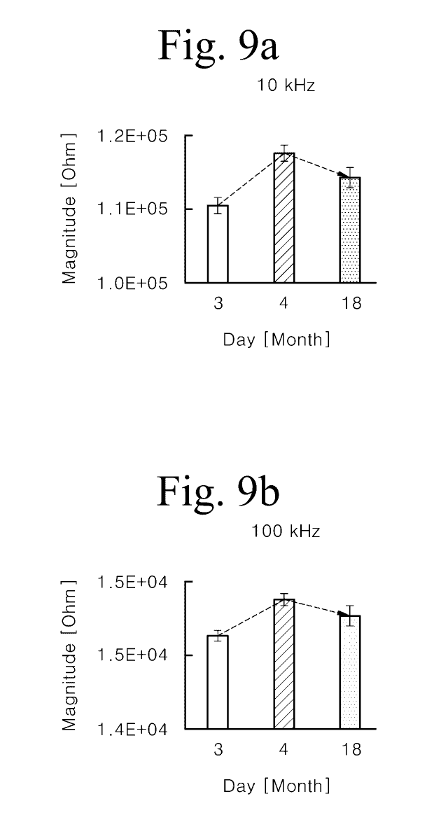

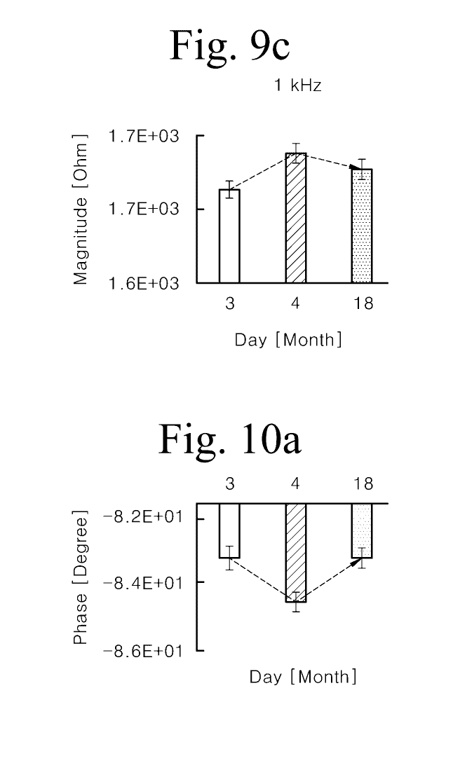

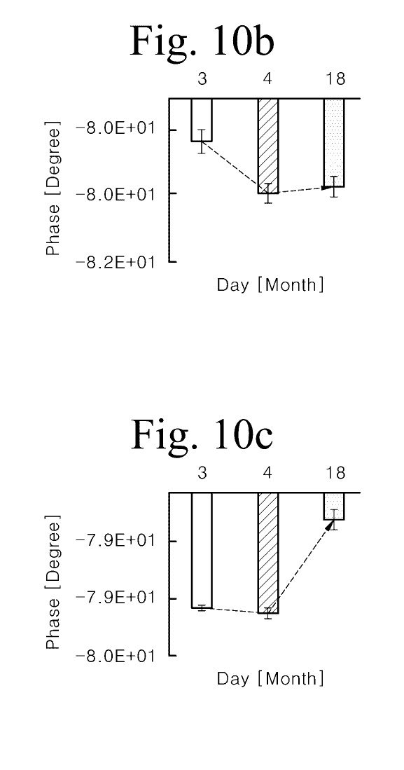

FIGS. 9a to 9c show the magnitude of electrical impedance, as measured on the first cell, the second cell, and the third cell at a frequency of 10 kHz, 100 kHz, and 1 MHz, respectively. FIGS. 10a to 10c show the phase of electrical impedance, as measured on the first cell, the second cell, and the third cell at a frequency of 10 kHz, 100 kHz, and 1 MHz, respectively (p<0.05, one-way ANOVA). It can be seen that, when the first cell grows to the second cell, the magnitude and phase of impedance increase, and, when the second cell grows to the third cell, the magnitude and phase of impedance decrease.

FIGS. 11a to 11c are views of an apparatus for measuring electrical properties of a biological sample according to another embodiment of the present invention.

Referring to FIGS. 11a to 11c, the apparatus for measuring electrical properties of a biological sample according to this embodiment may include: a first channel 2 through which a biological sample flows; and a second channel 4′ which is a negative pressure channel. The first channel 2 may be parallel to the second channel 4′. The first channel 2 may be provided therein with a biological sample trap and the first channel 2 may communicate with the second channel 4′ at a position at which the first channel 2 is provided with the biological sample trap.

Thus, a hole may be formed between the first channel 2 and the second channel 4′ to allow air to flow from the first channel 2 having higher pneumatic pressure toward the second channel 4′ having lower pneumatic pressure.

Thus, among biological samples flowing through the first channel 2, a single biological sample can be trapped by the biological sample trap due to the flow of fluid. In order to enhance such a trapping effect, barriers of the biological sample trap may have an inclined structure. Specifically, the barriers 5, 5′ may be inclined in a direction in which a biological sample can be slid from the first channel 2 toward the second channel 4′.

Electrodes 6 may be formed on the barriers 5, 5′. Each of the barriers may be a single barrier having a conical shape tapered toward the second channel 4′, or may include a plurality of barriers having a poly-pyramidal shape tapered toward the second channel 4′. The apparatus according to this embodiment may further include a pneumatic pressure channel, in addition to the negative pressure channel 4′.

The biological sample trap may include a plurality of biological sample traps, each of which is configured to trap a single biological sample such that electrical properties of the trapped biological sample can be measured.

Therefore, according to the present invention, it is possible to provide a more accurate technique for in-situ measurement at a single cell level.

Although some embodiments have been described herein, it should be understood by those skilled in the art that these embodiments are given by way of illustration only, and that various modifications, variations and alterations can be made without departing from the spirit and scope of the invention. Therefore, the embodiments and the accompanying drawings should not be construed as limiting the spirit of the present invention, but should be construed as illustrating the spirit of the present invention. The scope of the invention should be interpreted according to the following appended claims as covering all modifications or variations derived from the appended claims and equivalents thereof.

What is claimed is:

1. An apparatus for measuring electrical properties of a biological sample, comprising:
a first channel through which a biological sample flows;
a second channel formed above or below the first channel; and
a plurality of electrodes adjoining the first channel and measuring electrical properties of the biological sample.

2. The apparatus according to claim 1, wherein a portion of a surface of the first channel between the first channel and the second channel is formed of a flexible film.

3. The apparatus according to claim 2, wherein the flexible film is deformed by a pressure of the second channel.

4. The apparatus according to claim 2, wherein the flexible film is bent inwardly of the first channel with increasing pressure of the second channel.

5. The apparatus according to claim 2, wherein, as the flexible film is bent inwardly of the first channel, the electrodes are brought into contact with the biological sample in the first channel.

6. The apparatus according to claim 1, wherein the first channel is provided therein with a biological sample trap.

7. The apparatus according to claim 6, wherein the biological sample trap comprises a plurality of barriers separated from one another by a distance corresponding to a size of a single biological sample.

8. The apparatus according to claim 1, wherein the electrodes measure electrical properties of the biological sample, the electrical properties comprising at least one of amplitude, phase, reactance, resistance, conductance, and capacitance.

9. The apparatus according to claim 1, wherein the biological sample comprises at least one of a cell, a tissue, and an organ of a living organism.

10. The apparatus according to claim 6, wherein the first channel, the second channel, or the biological sample trap is formed by lithography and photoresist processes.

11. The apparatus according to claim 6, further comprising:
a controller regulating an pneumatic pressure of the second channel when a single biological sample is situated in the biological sample trap.

12. The apparatus according to claim 1, wherein the first channel is orthogonal to the second channel.

13. The apparatus according to claim 6, wherein the second channel is a negative pressure channel and the first channel communicates with the second channel at a position at which the biological sample trap is provided.

14. The apparatus according to claim 13, wherein the biological sample trap comprises a plurality of barriers inclined in a direction in which the first channel communicates with the second channel.

15. The apparatus according to claim 14, wherein the electrodes are formed on the barriers.

16. The apparatus according to claim 7, wherein the barriers have an inclined structure.

17. A system for measuring electrical properties of a biological sample, comprising:
the apparatus for measuring electrical properties of a biological sample according to claim 1;
an electrical property analyzer analyzing electrical properties measured by the apparatus; and
a pneumatic pump infusing air into the second channel.

18. The system according to claim 17, further comprising:
a display displaying results of measuring and analyzing the electrical properties.

19. The system according to claim 17, further comprising:
a computing device storing output values of the electrical property analyzer and assessing a degree of aging of the biological sample based on the stored output values.
